(19) United States
(12) Patent Application Publication
(10) Pub. No.: US 2019/0071653 A1
(43) Pub. Date: Mar. 7, 2019

(54) HALOACID DEHALOGENASE SUPERFAMILY PROTEIN VARIANT AND METHOD OF REDUCING CONCENTRATION OF FLUORINE-CONTAINING COMPOUND IN SAMPLE USING THE SAME

(71) Applicant: Samsung Electronics Co., Ltd., Suwon-si (KR)

(72) Inventors: Yukyung Jung, Hwaseong-si (KR); Taeyong Kim, Daejeon (KR); Jongwon Byun, Suwon-si (KR); Jinhwan Park, Suwon-si (KR)

(21) Appl. No.: 16/047,750

(22) Filed: Jul. 27, 2018

(30) Foreign Application Priority Data
Sep. 1, 2017 (KR) 10-2017-0111925

(51) Int. Cl. C12N 9/14

(52) U.S. Cl. CPC C12N 9/14; C12Y 308/01002

(57) ABSTRACT

Provided are a haloacid dehalogenase superfamily protein variant and a method of reducing a concentration of a fluorine-containing compound in sample using the same.

### CROSS-REFERENCE TO RELATED APPLICATION

This application claims the benefit of Korean Patent Application No. 10-2017-0111925, filed on Sep. 1, 2017, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 34,097 Byte ASCII (Text) file named “738072_ST25.TXT,” created on May 17, 2018.

### BACKGROUND

### 1. Field

The present disclosure relates to a recombinant microorganism, which includes a foreign gene encoding a variant of a haloacid dehalogenase superfamily protein, a composition including the variant for use in removing a fluorine-compound in a sample, and a method of reducing a concentration of a fluorine-containing compound in a sample using the haloacid dehalogenase superfamily protein.

### 2. Description of the Related Art

The emission of greenhouse gases, which have accelerated global warming, is a serious environmental problem, and regulations to reduce and prevent the emissions of greenhouse gases have been tightened. Among the greenhouse gases, fluorinated gases (F-gases), such as perfluorocarbons (PFCs), hydrofluorocarbons (HFCs), and sulfur hexafluoride (SF6), show low absolute emission, but have a long half-life and a very high global warming potential, resulting in significantly adverse environmental impact. The amount of F-gases emitted from the semiconductor and electronics industries, which are major causes of F-gas emission, has exceeded the assigned amount of greenhouse gas emissions and continues to increase. Therefore, costs required for decomposition of greenhouse gases and greenhouse gas emission allowances are increasing every year.

A pyrolysis or catalytic thermal oxidation process has generally been used in the decomposition of F-gases. However, this process has disadvantages of limited decomposition rate, emission of secondary pollutants, and high cost. However, biological decomposition of F-gases would allow F-gases to be treated in a more economical and environmentally-friendly manner.

Therefore, there is a need to develop new microorganisms and methods for the biological decomposition of F-gases. This invention provides such microorganisms and methods.

### SUMMARY

Provided is a variant haloacid dehalogenase superfamily protein, as well as a polynucleotide encoding the variant.

Also provided herein is a recombinant microorganism including a foreign gene encoding the variant haloacid dehalogenase superfamily protein.

Further provided is a composition for use in reducing a fluorine-containing compound in a sample, the composition including the variant haloacid dehalogenase superfamily protein or recombinant microorganism expressing same.

Also provided is a method of reducing a concentration of a fluorine-containing compound in a sample, the method including contacting the variant of a haloacid dehalogenase superfamily protein with a sample including a fluorine-containing compound, so as to reduce the concentration of the fluorine-containing compound in the sample, wherein the variant haloacid dehalogenase protein is, optionally, in a recombinant microorganism that expresses the protein.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a pET-BC3334 vector;

FIG. 2 is a schematic diagram of a glass Dimroth reflux condenser;

FIG. 3 is a vector map of a pET-SF0757 vector; and

FIG. 4 shows alignment results of homologous sequence of a BC3334 protein.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items.

The term “gene” as used herein refers to a polynucleotide that expresses a particular protein. A gene may include regulatory sequences, such as a coding region sequence and a non-coding region including a 5′ non-coding sequence and a 3′ non-coding sequence. The regulatory sequence may also include a promoter, an enhancer, an operator, a ribosome binding site, a polyA binding site, a terminator region, and the like.

The term “sequence identity” as used herein with respect to a nucleic acid or a polypeptide refers to a degree of identity between bases or amino acid residues of sequences after being aligned to best match in a certain comparative region. The sequence identity is a value measured by comparing two sequences in a certain comparative region through optimal alignment of the two sequences, wherein some portions of the sequences in the comparative region may be added or deleted compared to a reference sequence. A percentage of sequence identity may be for example, calculated as follows: two sequences that are optimally aligned are compared in the entire comparative region; the number of locations where the same amino acids or nucleic acids appear in both sequences is determined to the number of matching locations; the number of matching locations is divided by the total number of locations (i.e., the size of a range) in the comparative region; and the result of the division is multiplied by 100 to obtain the percentage of the sequence identity. The percentage of the sequence identity may be determined using a known sequence comparison program, such as BLASTN or BLASTP (NCBI), CLC Main Workbench (CLC bio), or MegAlign™ (DNASTAR Inc). Unless otherwise mentioned in the specification, the selection of the parameters used to execute the program may be as follows: E-value=0.00001 and H-value=0.001.

An aspect of the present invention provides a recombinant microorganism including a foreign gene encoding a variant of a haloacid dehalogenase (HAD) superfamily protein.

A HAD superfamily protein may be an enzyme including phosphatase, phosphonatase, P-type ATPase, beta-phosphoglucomutase, phosphomannomutase, and dehalogenase. The HAD superfamily protein may include a HAD domain. The HAD superfamily protein may be phospholipid-translocating ATPase belonging to EC 3.6.3.1, 3-deoxy-D-manno-octulosonate (KDO) 8-phosphate phosphatase belonging to EC 3.1.3.45, mannosyl-3-phosphoglycerate phosphatase belonging to EC 3.1.3.70, phosphoglycolate phosphatase belonging to EC 3.1.3.18, or HAD belonging to EC 3.8.1.2.

The ATPase may be a putative lipid-flipping enzyme involved in cold tolerance in Arabidopsis. The 3-deoxy-D-manno-octulosonate (KDO) 8-phosphate phosphatase may catalyze the final step in the biosynthesis of KDO, which is a component of lipopolysaccharide in Gram-negative bacteria. The mannosyl-3-phosphoglycerate phosphatase may hydrolyse mannosyl-3-phosphoglycerate to form osmolyte mannosylglycerate. The phosphoglycolate phosphatase may catalyze the dephosphorylation of 2-phosphoglycolate.

The HAD superfamily protein thereof may have a sequence identity of 85% or more, 90% or more, 95% or more, 96% or more, 97% or more, 98% or more, or 99% or more with respect to an amino acid sequence of SEQ ID NO: 1, 5, 29, 30, 31, 32, or 33.

The variant may be a HAD superfamily protein as described above with an amino acid alteration in at least one amino acid residue corresponding to positions N122 and S184, of SEQ ID NO: 1. Thus, for instance, the variant HAD superfamily protein may have a sequence identity of 85% or more, 90% or more, 95% or more, 96% or more, 97% or more, 98% or more, or 99% or more with respect to an amino acid sequence of SEQ ID NO: 1, 5, 29, 30, 31, 32, or 33 provided the sequence comprises alteration in one or more amino acid residues corresponding to N122 or S184. The variant itself may be an enzyme belonging to the HAD superfamily, for example, an enzyme having the activity of a dehalogenase belonging to EC 3.8.1.2. The amino acid alteration may include substitution of V or Y for N122, substitution of H, Q, or D for S184, or a combination thereof. Alternatively, the alteration may be a conservative substitution for V or Y at position 122, or a conservative substitution for H, Q, or D at position 184. In other words, the alteration may be a substitution of the amino acid corresponding to N122 with an amino acid that is conservative with respect to V or Y, or substitution of the amino acid corresponding to S184 with an amino acid that is conservative with respect to H, Q, or D. A substitution at S184 that is a conservative with respect to H may be S184K, or S184R. A substitution at S184 that is a conservative with respect to Q may be S184T, S184C, S184Y, or S184N. A substitution at S184 that is conservative with respect to D may be S184E. A substitution at N122 that is conservative with respect to V may be N122G, N122A, N122L, N122I, N122M, N122F, N122W, or N122P. A substitution at N122 that is conservative with respect to Y may be N122S, N122T, N122C, or N122Q.

The variant may be prepared by substituting at least one residue, which corresponds to N122 and S184 in the HAD superfamily protein BC3334 having the amino acid of SEQ ID NO: 1, with another amino acid, for example, one of the 19 natural amino acids. The variant may be prepared by substitution of V or Y for an amino acid residue corresponding to residue N122 position in BC3334 having amino acid sequence of SEQ ID NO: 1. The variant may be prepared by substitution of H, Q, or D for an amino acid residue corresponding to residue S184 of the amino acid sequence of SEQ ID NO: 1. The amino acid residues corresponding to the N122 position and the S184 position in BC3334 of the amino acid sequence of SEQ ID NO: 1 may each be an amino acid residue corresponding to the N122 position and an amino acid residue corresponding to the S184 position in SF0757 of an amino acid sequence of SEQ ID NO: 5.

The variant may have the substitution N122V or N122Y in the amino acid sequence of SEQ ID NO: 1, or in some embodiments, may have S184H, S184Q, or S184D, in the amino acid sequence of SEQ ID NO: 5. In other words, the variant can comprise SEQ ID NO: 1 in which N122 is substituted with V or Y, or SEQ ID NO: 5 in which S184 is substituted with H, Q, or D.

The variant may be a single variant, such as a N122V, N122Y, S184H, S184Q, or S184D (e.g., of any of SEQ ID NOs: 1, 5, 29, 30, 31, 32, or 33), or a double variant, such as N122V and S184H; N122V and S184Q; N122V and S184D; N122Y and S184H; N122Y and S184Q; or N122Y and S184D (e.g., of any of SEQ ID NOs: 1, 5, 29, 30, 31, 32, or 33). In some embodiments, the variant HAD protein comprises SEQ ID NO: 19, 21, 23, 25, or 27, or a sequence with 85%, 90%, 95%, or 99% sequence identity thereto.

The amino acid alteration may include substitution, insertion, or deletion. The substitution may include substitution with an amino acid that is modified after translation. The substitution may include substitution with one of 19 amino acids other than the corresponding amino acid among 20 natural amino acids. Amino acids used herein and abbreviations thereof are shown in Table 1.

TABLE 1

| Abbreviation | | Amino acid |
|---|---|---|
| A | Ala | Alanine |
| C | Cys | Cysteine |
| D | Asp | Aspartic acid |
| E | Glu | Glutamic acid |
| F | Phe | Phenylalamine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

The term “conservative” or “conservative substitution” as used herein refers to substitution of an amino acid with a similar amino acid in terms of the amino acid characteristics. For example, when a non-aliphatic amino acid residue (e.g., Ser) at a specific position is substituted with an aliphatic amino acid residue (e.g., Leu), a substitution with a different aliphatic amino acid (e.g., ILe or Val) at the same position is referred to as a conservative mutation. In addition, the amino acid characteristics include size of the residue, hydrophobicity, polarity, charge, pK-value, and other amino acid characteristics known in the art. Accordingly, a conservative mutation may include substitution, such as basic for basic, acid for acid, polar for polar, and the like. Conservative substitutions may be made, for example, according to Table 2 below which describes a generally accepted grouping of amino acid characteristics.

TABLE 2

| Set | Amino acids |
|---|---|
| Non-polar | G A V L I M F W P |
| Polar | S T C Y N Q |
| Acidic | D E |
| Basic | K R H |

The term “corresponding” as used herein refers to the amino acid position of a protein of interest that aligns with the mentioned position of a reference protein (e.g., position N122 or S184 of SEQ ID NO: 1) when amino acid sequences of the protein of interest and the reference protein are aligned using an art-acceptable protein alignment program, such as the BLAST pairwise alignment or the well known Lipman-Pearson Protein Alignment program. For example, the amino acid residues at the positions N122 and S184 of the amino acid sequence of SEQ ID NO: 1 may each correspond to the amino acid residue of a protein of interest, for example, position N122 and position S184 of the amino acid sequence of SEQ ID NO: 5. The protein of interest may be HAD, which belongs to, for example, EC 3.8.1.2. The database (DB) in which the reference sequence is stored may be Reference Sequence (RefSeq) non-redundant protein database of NCBI. The parameters used for the sequence alignment may be as follows: E-value 0.00001 and H-value 0.001.

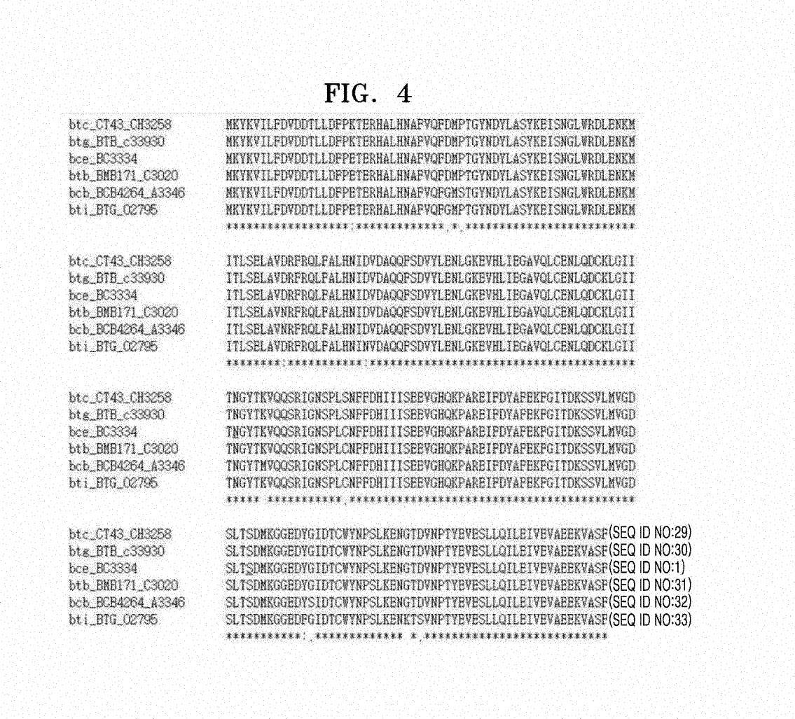

Examples of the proteins obtained according to the alignment conditions above and having the amino acid residues corresponding to positions N122 and S184 of the amino acid sequence of SEQ ID NO: 1 (hereinafter, referred to as “homologs of BC3334”) are shown in Table 3 below. The homologs may have 85% or more sequence identity to the amino acid sequence of SEQ ID NO: 1. The results of aligning the sequences and the numbering of the sequences are shown in FIG. 4. In FIG. 4, the underlined parts represent the positions N122 and S184, the N-terminal residue is 1, and the C-terminal residue is 236.

TABLE 3

| SEQ ID | Gene symbol | |
|---|---|---|
| NO: | (Locus tag) | Gene description |
| 29 | CT43_CH3258 | 2-haloalkanoic acid dehalogenase |
| | | Bacillus thuringiensis serovar chinensis |
| | | CT-43 |
| 30 | BTB_c33930 | yfnB1: tentative HAD-hydrolase YfnB |
| | | Bacillus thuringiensis Bt407 |
| 31 | BMB171_C3020 | 2-haloalkanoic acid dehalogenase |
| | | Bacillus thuringiensis BMB171 |
| 32 | BCB4264_A3346 | Hydrolase, HAD-like family |
| | | Bacillus cereus B4264 |
| 33 | BTG_02795 | 2-haloalkanoic acid dehalogenase |
| | | Bacillus thuringiensis HD-771 |

The recombinant microorganism may be bacteria or fungi, and the bacteria may be Gram-positive or Gram-negative. The Gram-negative bacteria may belong to the Enterobacteriaceae family. The Gram-negative bacteria may belong to the genus Escherichia, the genus Samonella, the genus Xanthobacter, or the genus Pseudomonas. The microorganism belonging to the genus Escherichia may be E. coli. The microorganism belonging to the genus Xanthobacter may be X. autotrophicus. The Gram-positive bacteria may belong to the genus Corynebacterium or the genus Bacillus. The recombinant microorganism may include at least one foreign or heterologous polynucleotide encoding a variant HAD superfamily protein as described herein, for example, a polynucleotide encoding SEQ ID NO: 19, 21, 23, 25, or 27, or having a nucleotide sequence of SEQ ID NO: 20, 22, 24, 26, or 28.

Another aspect of the invention provides a composition including a variant of a haloacid dehalogenase (HAD) superfamily protein, as described herein, for use in removing a fluorine-containing compound from a sample. In certain embodiments the composition may comprise a fluorine-containing compound, such as those described herein.

The fluorine-containing compound may be represented by Formula 1 or 2:

C(R¹)(R²)(R³)(R⁴) <Formula 1>

(R⁵)(R⁶)(R⁷)C—[C(R¹¹)(R¹²)]ₙ—C(R⁸)(R⁹)(R¹⁰) <Formula 2>

In Formulae 1 and 2, n may be an integer from 0 to 10; R¹, R², R³, and R⁴ may each independently be fluorine (F), chlorine (CI), bromine (Br), iodine (I), or hydrogen (H), provided at least one of R¹, R², R³, or R⁴ is F; R⁵, R⁶, R⁷, R⁸, R⁹, R¹⁰, R¹¹, and R¹² may each be independently F, Cl, Br, I, or H, provided that at least one of R⁵, R⁶, R⁷, R⁸, R⁹, R¹⁰, R¹¹, or R¹² is F.

The fluorine-containing compound may be, for example, CHF₃, CH₂F₂, CH₃F, or CF₄. The term “removal” as used herein refers to reduction of the concentration of the fluorine-containing compound in the sample, and the reduction includes partial or complete removal.

The variant of the HAD protein may be provided in the composition by a recombinant microorganism that expresses the variant, i.e., a recombinant microorganism that includes a foreign gene that encodes the variant of the HAD protein. The composition may include the recombinant microorganism itself, a lysate thereof, or an aqueous material fraction of the lysate. The recombinant microorganism for use in the composition may be any recombinant microorganism described herein.

The removal of the fluorine-containing compound from the sample encompasses any reduction of the concentration of the fluorine-containing compound in the sample, which may be achieved by cleavage of a C—F bond of the fluorine-containing compound, conversion of the fluorine-containing compound into a different material, or accumulation of the fluorine-containing compound in a cell. The conversion of the fluorine-containing compound may include introduction of a hydrophilic group, such as a hydroxyl group, to the fluorine-containing compound, or introduction of a carbon-carbon double bond or a carbon-carbon triple bond to the fluorine-containing compound.

The sample may be a liquid sample or a gaseous sample. The sample may be, for instance, industrial sewage or waste gas.

Another aspect of the disclosure provides a method of reducing a concentration of a fluorine-containing compound in a sample, the method including contacting the variant haloacid dehalogenase (HAD) superfamily protein described herein (or composition comprising same or microorganism expressing same) with a sample including a fluorine-containing compound, so as to reduce the concentration of the fluorine-containing compound in the sample. The term “removal” as used herein refers to reduction of the concentration of the fluorine-containing compound in the sample, and includes partial or complete removal.

Contacting of the variant HAD superfamily protein with the fluorine-containing sample may be performed in any manner. In one embodiment, the contacting is performed in an air-tight closed container. The contacting may be gas-liquid contacting of a gaseous sample with a liquid containing the variant of the HAD protein. In addition, the contacting may be liquid-liquid contacting of a liquid sample with a liquid containing the variant of the HAD protein. The contacting may include mixing.

The variant of the HAD protein for use in the inventive method may be included in a recombinant microorganism including a foreign gene that encodes the variant of the HAD protein. In this regard, the contacting may include contacting the sample with a cell first, and then, with the variant protein in the cell. The variant protein may be provided in the recombinant microorganism, a lysate thereof, or an aqueous material fraction of the lysate. Contacting the recombinant microorganism comprising the HAD superfamily protein with the sample comprising a fluorine-containing compound may be performed under conditions where the recombinant microorganism may survive in an air-tight closed container. Such conditions for the survival of the recombinant microorganism may include conditions where the recombinant microorganism may proliferate or conditions where the recombinant microorganism may be allowed to be in a resting state. In this regard, the contacting may include culturing a microorganism in the presence of the fluorine-containing compound. The culturing may be performed under aerobic or anaerobic conditions.

The sample of the inventive method may be a liquid sample or a gaseous sample. The sample may be industrial sewage or waste gas.

Another aspect of the invention provides a variant haloacid dehalogenase (HAD) superfamily protein itself. The variant protein can be any of the variant HAD superfamily proteins described herein with respect to the other aspects of the disclosure. Generally, the variant HAD protein includes an amino acid alteration in at least one amino acid residue corresponding to positions N122 and S184 of SEQ ID NO: 1.

Another aspect of the invention provides a polynucleotide encoding a variant haloacid dehalogenase (HAD) superfamily protein. The polynucleotide and the variant HAD protein encoded thereby can be any previously described herein with respect to the other aspects of the disclosure. The polynucleotide encoding the variant may be included in a vector. For use as a vector, any vehicle that can be used to introduce the polynucleotide to a microorganism may be used. The vector may be a plasmid vector or a viral vector. The vector may be in a recombinant microorganism as described herein.

Hereinafter, the present invention will be described in more detail with reference to Examples. However, these Examples are provided for illustrative purposes only, and the invention is not intended to be limited by these Examples.

### Example 1: Recombinant E. coli Expressing BC3334 Gene and Removal of a Fluorine-Containing Compound in a Sample Using the Recombinant E. coli

Recombinant E. coli expressing a HAD gene or a gene of a variant of the HAD gene was prepared, and the effect on the removal of CF₄ in a sample was confirmed.

1. Amplification of a HAD Gene (BC3334) from B. cereus and Introduction of the Gene into E. coli

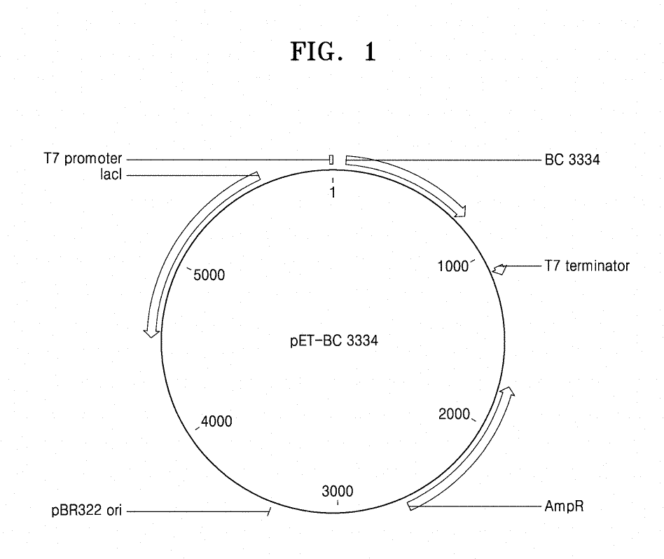

The BC3334 gene from B. cereus (ATCC 14579) was amplified. For amplification of the BC3334 gene, PCR was performed using the genomic DNA of the strain as a template and a set of primers having the nucleotide sequences of SEQ ID NOs: 3 and 4. The amplified BC3334 gene was ligated with a pETDuet-1 (Novagen, Cat. No. 71146-3), which was digested with restriction enzymes, NcoI and HindIII, using the InFusion Cloning Kit (Clontech Laboratories, Inc.), thereby preparing a pET-BC3334 vector. FIG. 1 is a vector map of the pET-BC3334. The BC3334 protein has an amino acid sequence of SEQ ID NO: 1, and is encoded by the nucleotide sequence of SEQ ID NO: 2.

Next, the pET-BC3334 vector was introduced into E. coli BL21 by a heat shock method, and then, cultured in an LB plate containing 100 μg/mL of ampicillin. Strains showing ampicillin resistance were selected. Then, a selected strain was designated as a recombinant E. coli BL21/pET-BC3334 wt.

2. Recombinant E. coli Expressing a Variant of the BC3334

A variant of the HAD protein BC3334 was prepared to improve the activity of the BC3334 protein on the removal of a fluorine-containing compound in a sample. Asparagine at position 122 (hereinafter, referred to as “N122”) of the amino acid sequence of SEQ ID NO: 1 and/or serine at position 184 (hereinafter, referred to as “S184”) was substituted with one of the other 19 natural amino acids. Here, each substitution is represented by “N122X” (wherein X indicates each of 19 natural amino acids other than asparagines) or “S184X” (wherein X indicates each of 19 natural amino acids other than serine). The effect of E. coli, which was prepared by introducing a gene encoding the prepared variant thereto, on the removal of CF₄ in a sample was confirmed.

The preparation of the N122X and/or S184X variant of SEQ ID NO: 1 was achieved by using the QuikChange II Site-Directed Mutagenesis Kit (Agilent Technology, USA). Site-directed mutagenesis using the kit was performed by using PfuUlta high-fidelity (HF) DNA polymerase for mutagenic primer-directed replication of two plasmid strands with the highest fidelity. The basic procedure utilizes a super-coiled double-stranded DNA (dsDNA) vector with an insert of interest and two synthetic oligonucleotide primers, both containing the desired mutation. The oligonucleotide primers, each complementary to opposite strands of the vector, were extended during temperature cycling by PfuUltra HF DNA polymerase, without primer displacement. Extension of the oligonucleotide primers generated a mutated plasmid containing staggered nicks. Following temperature cycling, the product was treated with DpnI. The DpnI endonuclease (target sequence: 5′-Gm⁶ATC-3′) was specific for methylated and hemimethylated DNA, and was used to digest the parental DNA template for the selection of mutation-containing synthesized DNA. Afterwards, the nicked vector DNA incorporating the desired mutations was then transformed into XL1-Blue supercompetent cells.

Among the primer sets used to induce mutagenesis of N122X and S184X, primer sets of SEQ ID NOs: 9 and 10 and primer sets of SEQ ID NOs: 11 and 12 were each used for N122Y and N122V. BC3334 proteins having the N122Y variation or having the N122V variation each had an amino acid sequence of SEQ ID NOs: 19 and 21, each of which amino acid sequences was encoded by a nucleotide sequence of SEQ ID NO: 20 and a nucleotide sequence of SEQ ID NO: 22.

In detail, PCR was performed by using the pET-BC3334 wt vector prepared in section (1) as a template and the primer sets for each of the variants as a primer, and a PfuUlta HF DNA polymerase to obtain variant vectors including staggered nicks. These vector products were treated with DpnI to select variant-containing synthesized DNA. Afterwards, the nicked vector DNA incorporating a desired variant was then transformed into XL1-Blue supercompetent cells, thereby cloning the pET-BC3334mt vector.

Lastly, the cloned pET-BC3334mt vector was introduced to a strain of E. coli BL21 in the same manner as in section (1), and a finally selected strain was designated as a recombinant E. coli BL21/pET-BC3334mt.

3. Effect of Recombinant E. coli Including a BC3334 Variant Introduced Thereto on the Removal of CF₄ in a Sample

The effect of the E. coli BL21/pET-BC3334mt prepared in section (2) and including the mutant BC3334 gene introduced thereto on the removal of CF₄ in a sample was confirmed.

In detail, a strain of the E. coli BL21/pET-BC3334mt was cultured in a LB medium with stirring at a temperature of 30□ at a speed of 230 rpm, and at an OD₆₀₀ of about 0.5, IPTG 0.2 mM was added to the medium, followed by being cultured overnight with stirring at a temperature of 20□ at a speed of 230 rpm. Then, the cells were harvested and suspended in a LB medium, so as to have a cell concentration OD₆₀₀ of 3.0. 10 mL of the cell solution was added to a 60 mL serum bottle, and the serum bottle was sealed. The LB medium was supplemented with 10 g of tripton per 1 L of distilled water, 5 g of an enzyme extract, and 10 g of NaCl. Next, CF₄ in a gas phase was injected into the serum bottle through a rubber stopper of a cap of the serum bottle with a syringe, so as to have 1,000 ppm of CF₄ in a head space of the serum bottle. Afterwards, the serum bottle was cultured for 4 days with stirring at a temperature of 30□ at a speed of 230 rpm. The experiments were performed in triplicate. After incubation, 0.5 mL of CF₄ gas was collected by using a 1.0 mL syringe from the head space, which did not contain the medium, of the serum bottle, and then, was injected into a gas chromatograph (GC) column (Agilent 7890, Palo Alto, Calif., USA). The injected CF₄ gas was separated by a CP-PoraBOND Q column (25 m length, 0.32 mm inner diameter, 5 um film thickness, Agilent), and changes in the concentration of CF₄ gas was analyzed by mass spectrometry (MS) (Agilent 5973, Palo Alto, Calif., USA). Here, helium was used as a carrier gas, and was flowed into the column at a rate of 1.5 ml/min. Regarding conditions for the GC, a temperature at an inlet was 250□, and an initial temperature was maintained at 40□ for 2 minutes and raised up to 290□ at a speed of 20□/min. Regarding conditions for the MS, an ionization energy was 70 eV, an interface temperature was 280□, an ion source temperature was 230□, and a quadrupole temperature was 150□. As a result, in Table 4, strains including the variant showed the activity of removing CF₄ gas in a sample as compared to a wild type.

TABLE 4

| | Decomposition rate of CF₄ (%, as |
|---|---|
| Strain | compared to a control group) |
| Wild type | 4.11 |
| BL21/pET- | 9.29 |
| BC3334mt(N122Y) | |
| BL21/pET- | 5.99 |
| BC3334mt(N122V) | |

In Table 4, the control group was E. coli BL21 to which an empty pETDuet vector was introduced instead of the pET-BC3334mt vector, and the wild type was E. coli including wild type BC3334 gene.

As shown in Table 4, the E. coli including the wild type BC3334 gene showed a reduction of CF₄ by 4.11% as compared to the control group, wherein the E. coli including the variants of the BC3334 gene, i.e., the N122Y and N122V genes, showed a reduction of CF₄ by 9.29% and 5.99%, respectively, as compared to the control group.

4. Decomposition of a Fluorine-Containing Compound by a Circulation Process

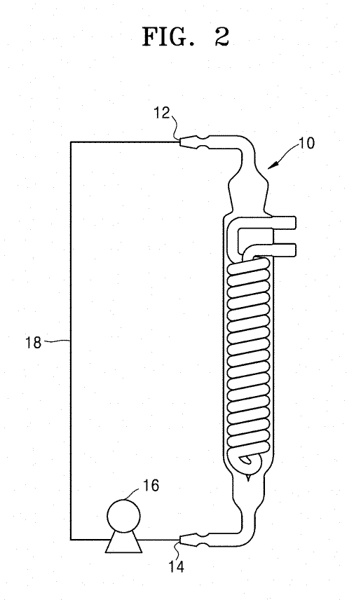

As shown in FIG. 2, 50 ml of an LB medium and 1,000 ppm of CF4 gas were added to a glass Dimroth coil reflux condenser (a reactor length: 350 mm, an exterior diameter: 35 mm, and an interior volume: 200 mL) that was sterilized and vertically oriented, and then, the LB medium was subjected to circulation. FIG. 2 is a schematic diagram of the glass Dimroth reflux condenser(10). The LB medium was supplied to an inlet(12) of an upper portion of the condenser(10), flowed through an inner wall of the condenser(10), and discharged to an outlet(14) of a lower portion of the condenser(10). The discharged LB medium was re-supplied to the inlet(10) along a circulation line(18). Although not shown in FIG. 2, to maintain the temperature, an inner screwed pipe of the condenser(10) was connected to a constant temperature zone of 30□. The circulation is performed by a pump (16). Here, the circulation rate of the LB medium was maintained at 4 mL/min. After an appointed period of time, i.e., 0, 48, 96, and 144 hours, the amount of the CF₄ gas in the condenser was confirmed by gas chromatography mass-spectrum (GC-MS). Then, it was confirmed that there was no change in the amount of CF₄ gas.

Subsequently, the recombinant microorganisms of sections (1) to (3) and the control group were each inoculated on an LB medium in the condenser(10) by using a syringe, so as to have an initial concentration of 5.0 on the basis of OD₆₀₀. The recombinant microorganism was E. coli to which a wild type BC3334 gene was introduced and E. coli to which a gene of the BC3334 variant was introduced. Then, the E. coli to which the wild type BC3334 gene was introduced and the E. coli to which the gene of the BC3334 variant was introduced were subjected to comparison of the CF₄ decomposition capability. The E. coli to which the empty vector was introduced was used as a negative control group, and there was no change in the level of CF₄.

Here, the circulation rate of the LB culture medium was about 4 mL/min, and the temperature inside the condenser(10) was maintained at 30□. After the strain inoculation and the elapse of 144 hours, the amount of CF₄ gas in the condenser(10) was confirmed by GC-MS. Then, the decomposition rate of CF₄ was calculated according to Equation 1, and the results are shown in Table 5.

Decomposition rate of CF₄=[(Initial amount of CF₄−amount of CF₄ after reaction)/initial amount of CF₄]×100 <Equation 1>

TABLE 5

| Strain | Decomposition rate of CF₄ (%) |
|---|---|
| BL21/pET-BC3334wt | 33.1 |
| BL21/pET-BC3334mt(N122Y) | 44.5 |

As shown in Table 5, the BC3334 variant after 144 hours of the culture showed an increase in the degradation rate by 1.34 times the degradation rate of the wild type strain, when applying the gas phase circulation process using the microorganism thereto.

### Example 2: Removal of CF₄ in a Sample by a Recombinant E. coli Expressing SF0757 Gene and a Gene of a Variant Thereof

1. Selection of a Strain of Bacillus bombysepticus SF3 and Decomposition of a Fluorine-Containing Compound by the Strain

In the present example, a microorganism capable of reducing a concentration of CF₄ in industrial wastewater was selected.

The sludge of the wastewater discharged from the plant of Samsung Electronics (Giheung, Korea) was smeared on an agar plate including a carbon-free agar plate (an agar medium supplemented with 0.7 g/L of K₂HPO₄, 0.7 g/L of MgSO₄.7H₂O, 0.5 g/L of (NH₄)₂SO₄, 0.5 g/L of NaNO₃, 0.005 g/L of NaCl, 0.002 g/L of FeSO₄.7H₂O, 0.002 g/L of ZnSO₄.7H₂O, 0.001 g/L of MnSO₄, and 15 g/L of agar), and the agar plate was added to a GasPak™ Jar (BD Medical Technology). The inside of the jar was filled with 99.9 v/v % CF₄, and the jar was sealed for the standing culture at a temperature of 30□ under anaerobic conditions. Single colonies formed after the culturing were cultured using a high throughput screening (HTS) system (Thermo Scientific/Liconic/Perkin Elmer). Each cultured single colony was inoculated on a 96-well microplate containing 100 μL of medium per well, and then, was subjected to static culture at a temperature of about 30□ for 96 hours under aerobic conditions. Meanwhile, the growth ability of the colonies was observed by measuring the absorbance at 600 nm every 12 hours.

The top 2% of strains showing excellent growth ability were selected and were each inoculated in a glass serum bottle (volume of 75 mL) containing 10 mL of the LB medium, so as to have an OD₆₀₀ of 0.5. The glass serum bottle was sealed, and then, CF₄ gas was injected thereto by using a syringe, so as to have 1,000 ppm of CF₄ gas. The glass serum bottle was incubated in a shaking incubator for 4 days at a temperature of 30□ with stirring at a speed of 230 rpm, and then, the amount of CF₄ in a head space was analyzed.

For the analysis, 0.5 ml of the headspace gas in the glass serum bottle was collected using a syringe, and then, the amount of CF₄ was analyzed under the same conditions as described in section (3). In the case of the control group, 1,000 of CF₄ having no cells was incubated and measured under the same conditions as described above.

Consequently, compared to the control group having no cells, the concentration of CF₄ was reduced by 10.27% in the separated microorganisms. The microorganisms had decomposition activity of 0.02586 g/kg-cell/h. To identify the selected strains, the genome sequences thereof were analyzed.

A genome obtained by assembling 3 contigs that were obtained by next generation sequencing (NGS) had a final size of 5.3 Mb, and as a result of gene annotation, a total of 5,490 genes were found to be present. As a result of phylogenetic tree analysis performed on each contig, it was confirmed that the microorganism belonged to Bacillus bombysepticus.

The separated microorganism was newly named as Bacillus bombysepticus SF3, deposited at the Korean Collection for Type Culture (KCTC), which is an international depository authority under the Budapest Treaty, on Feb. 24, 2017, and assigned the accession number of KCTC 13220BP.

2. Preparation of a Recombinant Microorganism Including a Gene Derived from a Strain of B. bombysepticus SF3 and a Variant Thereof

By the genomic sequence analysis of the strain of B. bombysepticus SF3 identified as described in section (1), genes presumed to encode dehalogenase, such as SF0757 (SEQ ID NO: 6), was selected.

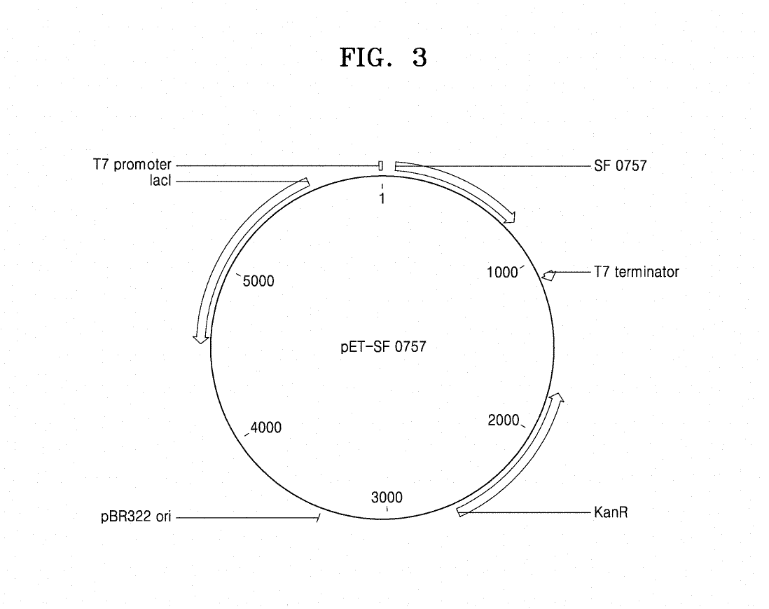

B. bombysepticus SF3 was cultured overnight in an LB medium with stirring at a temperature of 30□ at a speed of 230 rpm, and genomic DNA thereof was isolated using a total DNA extraction kit (Invitrogen Biotechnology). PCR was performed using the genome DNA as a template and a set of primers having nucleotide sequences of SEQ ID NOs: 7 and 8, as so to amplify a F0757 gene. The genes thus amplified were ligated with a pET28a vector (Novagen, Cat. No. 69864-3), respectively which was digested with restriction enzymes, such as NcoI and XhoI, by using an InFusion Cloning Kit (Clontech Laboratories, Inc.), so as to prepare a pET-SF0757 vector. FIG. 3 is a vector map of the pET-SF0757 vector. Here, the SF0757 gene had a nucleotide sequence of SEQ ID NO: 6, and encoded an amino acid sequence of SEQ ID NO: 5.

Next, the prepared pET-SF0757 vector was introduced to E. coli BL21 by a heat shock method, and then, cultured in an LB plate agar supplemented with 50 μg/mL of kanamycin. Strains showing kanamycin resistance were then selected, and a finally selected strain was designated as a recombinant E. coli BL21/pET-SF0757.

3. Preparation of a Recombinant E. coli Expressing a SF0757 Variant

In this section, a variant was prepared to improve the activity of the SF0757 gene on the removal of a fluorine-containing compound in a sample. Asparagine at position 122 (hereinafter, referred to as “N122”) of the amino acid sequence of SEQ ID NO: 5 and/or serine at position 184 (hereinafter, referred to as “S184”) was substituted with other 19 natural amino acids. Here, each substitution is represented by “N122X” (wherein X indicates each of 19 natural amino acids other than asparagines) or “S184X” (wherein X indicates each of 19 natural amino acids other than serine). The effect of E. coli, which was prepared by introducing a gene encoding the prepared variant thereto, on the removal of CF₄ in a sample was confirmed.

Among the primer sets used to induce mutagenesis of N122X and S184X, primer sets of SEQ ID NOs: 13 and 14, primer sets of SEQ ID NOs: 15 and 16, and primer sets of SEQ ID NOs: 17 and 18 were used for S184H, S184Q, and S184D. The SF0757 protein having the S184H, S184Q, and S184D variants each had an amino acid sequence of SEQ ID NOs: 23, 25, and 27, each of which amino acid sequences was encoded by a nucleotide sequence of SEQ ID NOs: 24, 26, and 28.

The preparation of the variant and the recombinant E. coli including a gene of the variant are the same as described in section (3) of Example 1.

Lastly, the cloned pET-SF0757mt vector was introduced to a strain of E. coli BL21 in the same manner as in section (1), and a finally selected strain was designated as a recombinant E. coli BL21/pET-SF0757mt.

4. Effect of Recombinant E. coli Including a SF0757 Variant Introduced Thereto on the Removal of CF₄ in a Sample

The influence of the E. coli BL21/pET-SF0757mt prepared in section (3) to which the SF0757 variant was introduced on the removal of CF₄ in a sample.

In detail, a strain of E. coli BL21/pET-SF0757mt was cultured in an LB medium with stirring at a temperature of 30□ at a speed of 230 rpm, and at an OD₆₀₀ of about 0.5, IPTG 0.2 mM was added to the medium, followed by being cultured overnight with stirring at a temperature of 20□ at a speed of 230 rpm. Then, the cells were harvested and suspended in a LB medium, so as to have a cell concentration OD₆₀₀ of 3.0. 10 mL of the cell solution was added to a 60 mL serum bottle, and the serum bottle was sealed. Next, CF₄ in a gas phase was injected to the serum bottle through a rubber stopper of a cap of the serum bottle by using a syringe, so as to have 1,000 ppm of CF₄ in a head space of the serum bottle. Afterwards, the serum bottle was cultured for 4 days with stirring at a temperature of 30□ at a speed of 230 rpm. The experiments were performed in triplicate. After incubation, CF₄ gas was collected from the head space, which did not contain the medium, of the serum bottle, and then, analyzed under the same conditions as described in section (3) of Example 1.

Consequently as shown in Table 6, the strains including the variants above exhibited increased activity of removing CF₄ gas in the sample as compared to the wild type strain.

TABLE 6

| | Decomposition of CF₄ (%, as |
|---|---|
| Strain | compared to control group) |
| Wild type | 3.95 |
| BL21/pET-SF0757(S184H) | 8.76 |
| BL21/pET-SF0757(S184Q) | 7.35 |
| BL21/pET-SF0757(S184D) | 5.61 |

In Table 6, the control group was E. coli BL21 to which an empty pET28a vector was introduced instead of the pET-SF0757 vector, and the wild type was E. coli including SF0757.

As shown in Table 6, E. coli including the SF0757 wild type gene showed a decrease in the level of CF₄ by 3.95% as compared to the control group, and the SF0757 variant, i.e., E. coli including the S184H, S184Q, and S184D genes, showed a decrease in the level of CF₄ by 8.76%, 7.35%, and 5.61%, respectively, as compared to the control group.

5. Decomposition of a Fluorine-Containing Compound Using a Circulation Process

The decomposition rate of the fluorine-containing compound in a sample was measured in the same manner as in section (4) of Example 1, except that strains of the recombinant E. coli BL21/pET-SF0757 prepared in section (3) were used.

TABLE 7

| Strain | Decomposition rate of CF₄ (%) |
|---|---|
| BL21/pET-SF0757wt | 25.7 |
| BL21/pET-SF0757mt(S184H) | 29.9 |

As shown in Table 7, the SF0757 variant after 144 hours of the culture showed an increase in the degradation rate by 1.16 times the degradation rate of the wild type strain, when applying the gas phase circulation process using the microorganism thereto.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A variant haloacid dehalogenase (HAD) superfamily protein, wherein the variant protein comprises an amino acid alteration in at least one amino acid residue corresponding to positions N122 and S184 of SEQ ID NO: 1.

2. A polynucleotide encoding the variant haloacid dehalogenase (HAD) superfamily protein of claim 1.

3. A recombinant microorganism comprising a foreign gene encoding a variant a haloacid dehalogenase (HAD) superfamily protein of claim 1.

4. The microorganism of claim 3, wherein the amino acid alteration comprises substitution of V or Y for N122 or substitution of a different amino acid for N122 that is conservative with respect to V or Y, wherein the substitution of a different amino acid for N122 that is conservative with respect to V is N122G, N122A, N122L, N122I, N122M, N122F, N122W, or N122P, and the substitution of a different amino acid for N122 that is conservative with respect to Y is N122S, N122T, N122C, or N122Q.

5. The microorganism of claim 3, wherein the amino acid alteration comprises substitution of H, Q, or D for S184 or substitution of a different amino acid for S184 that is conservative with respect to H, Q, or D, wherein the substitution of a different amino acid for S184 that is conservative with respect to H is S184K, or S184R, the substitution of a different amino acid for S184 that is conservative with respect to Q is S184T, S184C, S184Y, or S184N and the substitution of a different amino acid for S184 that is conservative with respect to D is S184E.

6. The microorganism of claim 3, wherein the HAD superfamily protein comprises an amino acid sequence having 85% or more sequence identity to SEQ ID NO: 1, 5, 29, 30, 31, 32, or 33.

7. The microorganism of claim 3, wherein the variant HAD superfamily protein comprises an amino acid sequence having 85% or more sequence identity to SEQ ID NO: 1, 5, 29, 30, 31, 32, or 33, and comprises substitution of at least one of N122 and S184 of SEQ ID NO: 1, 5, 29, 30, 31, 32, or 33.

8. The microorganism of claim 3, wherein the variant comprises SEQ ID NO: 1 with an N122Y substitution, SEQ ID NO: 5 with an S184H substitution, or SEQ ID NO: 1 or SEQ ID NO: 5 comprising both an N122Y and S184H substitution.

9. The microorganism of claim 3, wherein the variant HAD protein comprises SEQ ID NO: 19, 21, 23, 25, or 27.

10. The microorganism of claim 3, wherein the microorganism is Escherichia.

11. A composition comprising
(a) the variant haloacid dehalogenase (HAD) superfamily protein of claim 1; and
(b) a fluorine-containing compound of Formula 1 or Formula 2:
C(R¹)(R²)(R³)(R⁴) <Formula 1>
(R⁵)(R⁶)(R⁷)C—[C(R¹¹)(R¹²)]n-C(R⁸)(R⁹)(R¹⁰), <Formula 2>
wherein, in Formulae 1 and 2,
n is an integer from 0 to 10;
R¹, R², R³, and R⁴ are each independently fluorine (F), chlorine (CI),
bromine (Br), iodine (I), or hydrogen (H), wherein at least one of R¹, R², R³, or R⁴ is F; and
R⁵, R⁶, R⁷, R⁸, R⁹, R¹⁰, R¹¹, and R¹² are each independently F, Cl, Br, I, or H, wherein at least one of R⁵, R⁶, R⁷, R⁸, R⁹, R¹⁰, R¹¹, or R¹² is F.

12. The composition of claim 11, wherein the amino acid alternation comprises substitution of V or Y for N122 or substitution of a different amino acid for N122 that is conservative with respect to V or Y, wherein the substitution of a different amino acid for N122 that is conservative with respect to V is N122G, N122A, N122L, N122I, N122M, N122F, N122W, or N122P, and the substitution of a different amino acid for N122 that is conservative with respect to Y is N122S, N122T, N122C, or N122Q.

13. The composition of claim 11, wherein the amino acid alternation comprises substitution of H, Q, or D for S184 or substitution of a different amino acid for S184 that is conservative with respect to H, Q, or D, wherein the substitution of a different amino acid for S184 that is conservative with respect to H is S184K, or S184R, the substitution of a different amino acid for S184 that is conservative with respect to Q is S184T, S184C, S184Y, or S184N and the substitution of a different amino acid for S184 that is conservative with respect to D is S184E.

14. The composition of claim 11, wherein the composition comprises a recombinant microorganism comprising a foreign gene that expresses the variant.

15. The composition of claim 14, wherein the microorganism belongs to the genus Escherichia.

16. The composition of claim 11, wherein the variant HAD superfamily protein comprises an amino acid sequence having 85% or more sequence identity to SEQ ID NO: 1, 5, 29, 30, 31, 32, or 33, and comprises substitution in at least one of N122 and S184 of SEQ ID NO: 1, 5, 29, 30, 31, 32, or 33.

17. A method of reducing a concentration of a fluorine-containing compound in a sample, the method comprising:
contacting the variant haloacid dehalogenase (HAD) superfamily protein of claim 1 with a sample comprising a fluorine-containing compound represented by Formula 1 or Formula 2, so as to reduce the concentration of the fluorine-containing compound in the sample:
C(R¹)(R²)(R³)(R⁴) <Formula 1>
(R⁵)(R⁶)(R⁷)C—[C(R¹¹)(R¹²)]ₙ—C(R⁸)(R⁹)(R¹⁰), <Formula 2>
wherein, in Formulae 1 and 2,
n is an integer from 0 to 10;
R¹, R², R³, and R⁴ are each independently fluorine (F), chlorine (CI), bromine (Br), iodine (I), or hydrogen (H), wherein at least one of R¹, R², R³, or R⁴ is F; and
R⁵, R⁶, R⁷, R⁸, R⁹, R¹⁰, R¹¹, and R¹² are each independently F, Cl, Br, I, or H, wherein at least one of R⁵, R⁶, R⁷, R⁸, R⁹, R¹⁰, R¹¹, or R¹² is F.

18. The method of claim 17, wherein the amino acid alternation comprises substitution of V or Y for N122 or substitution of a different amino acid for N122 that is conservative with respect to V or Y, wherein the substitution of a different amino acid for N122 that is conservative with respect to V is N122G, N122A, N122L, N122I, N122M, N122F, N122W, or N122P, and the substitution of a different amino acid for N122 that is conservative with respect to Y is N122S, N122T, N122C, or N122Q.

19. The method of claim 17, wherein the amino acid alternation comprises substitution of H, Q, or D for S184 or substitution of a different amino acid for S184 that is conservative with respect to H, Q, or D, wherein the substitution of a different amino acid for S184 that is conservative with respect to H is 5184K, or S184R, the substitution of a different amino acid for S184 that is conservative with respect to Q is 5184T, 5184C, 5184Y, or S184N and the substitution of a different amino acid for S184 that is conservative with respect to D is S184E.

20. The method of claim 17, wherein the variant HAD superfamily protein comprises an amino acid sequence having 85% or more sequence identity to SEQ ID NO: 1, 5, 29, 30, 31, 32, or 33, and comprises substitution in at least one of N122 and S184 of SEQ ID NO: 1, 5, 29, 30, 31, 32, or 33.

21. The method of claim 17, wherein the variant HAD protein is in a recombinant microorganism comprising a foreign gene that expresses the variant HAD protein.

22. The method of claim 21, wherein the contacting the variant HAD protein with the sample comprising the fluorine-containing compound comprises culturing the microorganism with the sample.


SEQUENCE LISTING (33 sequences)

SEQ ID NO: 1 - protein, 236 aa, Bacillus cereus
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITNGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 2 - DNA, 711 bp, Bacillus cereus
atgaaatacaaagttatattattcgacgtagatgatacattattagatttccctgaaacggaaagacacgcattacataatgcgtttgtacagtttgatatgcctacagggtataatgattatcttgcaagctataaagagattagtaatggattatggagagatttagaaaataaaatgattacgctaagtgaattagcagtagatcgatttagacaattatttgcacttcataatatagacgtagatgcacagcaatttagtgatgtataccttgaaaatttagggaaggaagtacatcttatagaaggcgcagtacaattatgtgaaaatctacaagattgcaagttaggtattattacgaatggatatacgaaggtgcaacaatcaagaatcggaaattcacctttatgtaatttctttgatcacattattatttctgaagaagttggtcatcaaaaaccagcacgtgagatttttgattatgcgtttgagaagtttgggattactgataaatcaagcgtactaatggttggagattcgttaacttctgatatgaaaggcggagaagattacggcattgatacgtgttggtataatccgagtttgaaagaaaacgggacagatgttaacccgacttatgaagtggagagtctgctccaaattttagaaattgtagaagtggcggaagaaaaggtagcttcattttaa

SEQ ID NO: 3 - DNA, 40 bp, Artificial Sequence
  Synthetic primer
aagaaggagatataccatgaaatacaaagttatattattc

SEQ ID NO: 4 - DNA, 41 bp, Artificial Sequence
  Synthetic primer
gcattatgcggccgcaagctttaaaatgaagctaccttttc

SEQ ID NO: 5 - protein, 236 aa, Bacillus bombysepticus
  misc_feature (1)..(236): SF3
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSSDMRGGEDYGIDTCWYNPSLKENRTDVKPSYEVESLLQILEIVEVTKEKVASF

SEQ ID NO: 6 - DNA, 711 bp, Bacillus bombysepticus
  misc_feature (1)..(711): SF3
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttcttctgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 7 - DNA, 37 bp, Artificial Sequence
  Synthetic primer
aagaaggagatataccatgaaatacaaatttatatta

SEQ ID NO: 8 - DNA, 39 bp, Artificial Sequence
  Synthetic primer
ggtggtggtggtgctcgattaaaatgaagctactttttc

SEQ ID NO: 9 - DNA, 39 bp, Artificial Sequence
  Synthetic primer
aaactgggcattatcacctatggttataccaaagtgcag

SEQ ID NO: 10 - DNA, 39 bp, Artificial Sequence
  Synthetic primer
ctgcactttggtataaccataggtgataatgcccagttt

SEQ ID NO: 11 - DNA, 39 bp, Artificial Sequence
  Synthetic primer
aaactgggcattatcaccgtgggttataccaaagtgcag

SEQ ID NO: 12 - DNA, 39 bp, Artificial Sequence
  Synthetic primer
ctgcactttggtataacccacggtgataatgcccagttt

SEQ ID NO: 13 - DNA, 39 bp, Artificial Sequence
  Synthetic primer
gttggagattcgctttctcatgatatgagaggcggagaa

SEQ ID NO: 14 - DNA, 39 bp, Artificial Sequence
  Synthetic primer
ttctccgcctctcatatcatgagaaagcgaatctccaac

SEQ ID NO: 15 - DNA, 39 bp, Artificial Sequence
  Synthetic primer
gttggagattcgctttctcaggatatgagaggcggagaa

SEQ ID NO: 16 - DNA, 39 bp, Artificial Sequence
  Synthetic primer
ttctccgcctctcatatcctgagaaagcgaatctccaac

SEQ ID NO: 17 - DNA, 39 bp, Artificial Sequence
  Synthetic primer
gttggagattcgctttctgatgatatgagaggcggagaa

SEQ ID NO: 18 - DNA, 39 bp, Artificial Sequence
  Synthetic primer
ttctccgcctctcatatcatcagaaagcgaatctccaac

SEQ ID NO: 19 - protein, 236 aa, Artificial Sequence
  Synthetic BC3334 N122Y mutant
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITYGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 20 - DNA, 711 bp, Artificial Sequence
  Synthetic BC3334 N122Y mutant
atgaaatataaagttattctgtttgacgttgatgacaccctgctggatttcccggaaaccgaacgtcacgcgctgcataacgcctttgttcagttcgatatgccgacgggttacaacgactatctggcgtcttacaaagaaatctccaacggcctgtggcgtgatctggaaaacaaaatgatcactctgtctgaactggctgttgaccgtttccgccagctgttcgctctgcacaacatcgacgtggacgcgcagcagttttctgatgtgtacctggagaacctgggtaaagaggttcacctgatcgaaggcgcagtacaactgtgtgaaaatttgcaggactgcaaactgggcattatcacctatggttataccaaagtgcagcagtcccgtatcggcaacagcccgctgtgcaacttctttgatcacatcatcatcagcgaagaagtcggtcaccagaaaccggcgcgtgaaatcttcgactacgcattcgaaaaattcggtatcaccgataaatccagcgtgctgatggtgggtgactctctgaccagcgatatgaaaggcggcgaagattacggtattgatacctgctggtacaatccgagcctgaaagaaaatggcaccgacgttaacccgacctacgaagtggaaagcctgctgcagatcctggaaattgttgaagttgctgaagagaaagtcgcttccttctaa

SEQ ID NO: 21 - protein, 236 aa, Artificial Sequence
  Synthetic BC3334 N122V mutant
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITVGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 22 - DNA, 711 bp, Artificial Sequence
  Synthetic BC3334 N122V mutant
atgaaatataaagttattctgtttgacgttgatgacaccctgctggatttcccggaaaccgaacgtcacgcgctgcataacgcctttgttcagttcgatatgccgacgggttacaacgactatctggcgtcttacaaagaaatctccaacggcctgtggcgtgatctggaaaacaaaatgatcactctgtctgaactggctgttgaccgtttccgccagctgttcgctctgcacaacatcgacgtggacgcgcagcagttttctgatgtgtacctggagaacctgggtaaagaggttcacctgatcgaaggcgcagtacaactgtgtgaaaatttgcaggactgcaaactgggcattatcaccgtgggttataccaaagtgcagcagtcccgtatcggcaacagcccgctgtgcaacttctttgatcacatcatcatcagcgaagaagtcggtcaccagaaaccggcgcgtgaaatcttcgactacgcattcgaaaaattcggtatcaccgataaatccagcgtgctgatggtgggtgactctctgaccagcgatatgaaaggcggcgaagattacggtattgatacctgctggtacaatccgagcctgaaagaaaatggcaccgacgttaacccgacctacgaagtggaaagcctgctgcagatcctggaaattgttgaagttgctgaagagaaagtcgcttccttctaa

SEQ ID NO: 23 - protein, 236 aa, Artificial Sequence
  Synthetic SF0757 S184H mutant
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSHDMRGGEDYGIDTCWYNPSLKENRTDVKPSYEVESLLQILEIVEVTKEKVASF

SEQ ID NO: 24 - DNA, 711 bp, Artificial Sequence
  Synthetic SF0757 S184H mutant
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcatgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 25 - protein, 236 aa, Artificial Sequence
  Synthetic SF0757 S184Q mutant
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSQDMRGGEDYGIDTCWYNPSLKENRTDVKPSYEVESLLQILEIVEVTKEKVASF

SEQ ID NO: 26 - DNA, 711 bp, Artificial Sequence
  Synthetic SF0757 S184Q mutant
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctcaggatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 27 - protein, 236 aa, Artificial Sequence
  Synthetic SF0757 S184D mutant
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSDDMRGGEDYGIDTCWYNPSLKENRTDVKPSYEVESLLQILEIVEVTKEKVASF

SEQ ID NO: 28 - DNA, 711 bp, Artificial Sequence
  Synthetic SF0757 S184D mutant
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttctgatgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 29 - protein, 236 aa, Bacillus cereus
MKYKVILFDVDDTLLDFPKTERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITNGYTKVQQSRIGNSPLSNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 30 - protein, 236 aa, Bacillus cereus; identical to SEQ ID NO: 29

SEQ ID NO: 31 - protein, 236 aa, Bacillus cereus
MKYKVILFDVDDTLLDFPETERHALHNAFVQFDMPTGYNDYLASYKEISNGLWRDLENKMITLSELAVNRFRQLFALHNIDVDAQQFSDVYLENLGKEVHLIEGAVQLCENLQDCKLGIITNGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 32 - protein, 236 aa, Bacillus cereus
MKYKVILFDVDDTLLDFPETERHALHNAFVQFGMSTGYNDYLASYKEISNGLWRDLENKMITLSELAVNRFRQLFALHNIDVDAQQFSDVYLENLGKEVHLIEGAVQLCENLQDCKLGIITNGYTMVQQSRIGNSPLCNFFDHIIISEEVGHQKPAREIFDYAFEKFGITDKSSVLMVGDSLTSDMKGGEDYSIDTCWYNPSLKENGTDVNPTYEVESLLQILEIVEVAEEKVASF

SEQ ID NO: 33 - protein, 236 aa, Bacillus cereus
MKYKVILFDVDDTLLDFPETERHALHNAFVQFGMPTGYNDYLASYKEISNGLWRDLENKMITLSELAVDRFRQLFALHNINVDAQQFSDVYLENLGKEVHLIEGAVQLCENLQDCKLGIITNGYTKVQQSRIGNSPLCNFFDHIIISEEVGHQKPAREIFDYAFEKFGITDKSSVLMVGDSLTSDMKGGEDFGIDTCWYNPSLKENKTSVNPTYEVESLLQILEIVEVAEEKVASF